(19) United States
(12) Patent Application Publication
(10) Pub. No.: US 2003/0022794 A1
(43) Pub. Date: Jan. 30, 2003

(54) Novel quaternary salts and their use in agricultural formulations

(76) Inventors: Robert D. Wysong, Wilmington, DE (US); Chia-Chung Chen, Nantou (TW); Chuen-Ing Tseng, Lawrenceville, NJ (US); Arturo Aclaro Tirol, Kendall Park, NJ (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/235,445

(22) Filed: Sep. 5, 2002

Related U.S. Application Data
(63) Continuation of application No. 10/024,752, filed on Dec. 19, 2001, now abandoned.
Application No. 10/024,752 is a continuation of application No. 09/685,670, filed on Oct. 10, 2000, now abandoned.
Application No. 09/685,670 is a continuation of application No. 09/315,182, filed on May 20, 1999, now abandoned.
Application No. 09/315,182 is a continuation of application No. PCT/US97/21383, filed on Nov. 19, 1997.

(51) Int. Cl. A01N 47/36; C07D 239/02

(52) U.S. Cl. 504/212; 504/214; 544/210; 544/327

(57) ABSTRACT

Novel agrichemical quaternary salts, compositions and a method to prepare them are disclosed.

### FIELD OF THE INVENTION

The present invention relates to novel agrichemical quaternary salts, compositions of said salts and a method for their preparation.

### BACKGROUND OF THE INVENTION

Bioactive pesticides in order to be usefully applied must be formulated. Typically, the bioactive pesticides can be prepared in liquid formulations for reasons of economy, but suffer from a number of problems including chemical instability of the bioactive pesticide or physical instability (e.g., sedimentation of the bioactive pesticide) and these problems have limited their potential. In the case of bioactive herbicides, these liquid formulations are not as stable as would be desired. More particularly, sulfonylurea herbicides which are a well-known, extremely potent class of herbicides generally consisting of a sulfonylurea bridge, —SO₂NHCONH— linking two aromatic or heteroaromatic rings, and various salts of said sulfonylureas lack stable, convenient, economical liquid formulations which has limited the commercial utility of sulfonylurea herbicides as well as salts thereof.

Sulfonylureas and formulations thereof are known to have herbicidal and plant growth regulant activity. Such formulations are useful as pre- or post-emergent herbicides and plant growth regulants, especially where homogenous liquid formulations are particularly advantageous. Liquid sulfonylurea or salts thereof in formulations are unstable and decomposed by water or nucleophilic reactant impurities in both aqueous and organic solvents. Maintaining the stability of a formulation of sulfonylureas is extremely important since an unstable formulation will be considerably less effective when utilized.

Methods of stabilizing sulfonylureas are known in the literature. For instance, U.S. Pat. No. 4,936,900 discloses suspending sulfonylureas or agricultural suitable salts of sulfonylureas in water saturated with carboxylate salts (e.g., ammonium, substituted ammonium, or alkali metal) or inorganic acid salts (e.g., phosphate) so as to achieve chemically stable suspensions at pH 6-10. The salts of the present invention are not disclosed.

### SUMMARY OF THE INVENTION

It has been discovered that the stability of liquid formulations of various pesticides can be enhanced by utilizing the quaternary ammonium salts of this invention. The pesticides that are within the scope of the invention are those pesticides that are unstable in liquid formulations. Among such pesticides the sulfonyl urea herbicides arc referred. The sulfonylurea quaternary salts of this invention, when compared to the disclosed sulfonylureas, impart higher solubility in water or in organic solvents (depending on the chosen quaternary salt) and enhanced storage stability.

Accordingly, this invention involves novel agrichemical quaternary salts, of pesticides which allow chemically stable homogenous solution formulations of the pesticide in hydrophobic liquids and in water to be prepared for the first time (e.g. as emulsifiable concentrate formulations or oils which spontaneously spread over the surface of the water in a rice paddy).

The novel quaternary salts of pesticides are compounds represented by Formula I

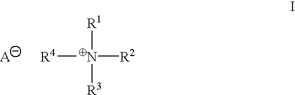

wherein R¹ is a substituted or unsubstituted benzyl or substituted or unsubstituted methylnapthyl group and the remaining groups R², R³ and R⁴ are substituted or unsubstituted C₁-C₂₅ alkyl or a substituted or unsubstituted alkaryl group; and A is defined as any known bioactive pesticide with a pKa from about 3 to about 7, provided that when the pesticide A is a sulfonylurea herbicide of the formula JSO₂NHCONR⁶Q and R¹ is unsubstituted benzyl, then one of R², R³ or R⁴ is other than methyl or ethyl.

Representative examples of the substituted or unsubstituted benzyl or substituted or unsubstituted methylnaphthyl groups of R¹ include but are not limited to

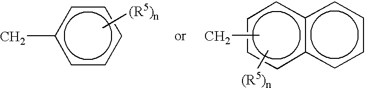

where R⁵ is C₁-C₃ alkyl, C₁-C₃ alkoxy or halogen, and n is 0-2.

Representative examples of substituted C₁-C₂₅ alkyl groups include but are not limited to C₁-C₂₅ haloalkyl and C₁-C₂₅ alkyl substituted with C₁-C₃ alkoxy, NO₂ or CN.

Representative examples of substituted or unsubstituted alkaryl groups include but are not limited to

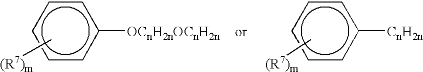

where R⁷ is C₁-C₁₂ alkyl, C₁-C₃ alkoxy, NO₂ or CN, m is 0 or 1, n is 1, 2 or 3.

The preferred alkaryl groups are

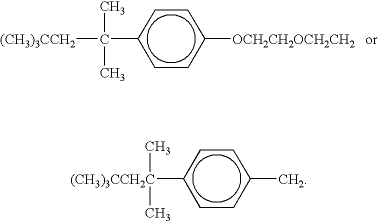

Representative examples of pesticides with a pKa from about 3 to about 7 include but are not limited to herbicides, insecticides and fungicides. Representative examples of herbicides include but are not limited to ureas, sulfonylureas, glyphosate and (2,4-dichlorophenoxy)acetic acid (2,4D). Representative examples of insecticides include but are not limited to methomyl and oxamyl. Representative examples of fungicides include but are not limited to carbendazim, flusilazole and cymoxanil.

The preferred pesticides of the invention are sulfonylurea herbicides wherein “A” of Formula I is J SO₂NH CONR⁶ Q where J is substituted or unsubstituted phenyl or heterocyclic group, Q is substituted or unsubstituted pyrimidine or substituted or unsubstituted triazine group and R⁶ is H or methyl.

Representative examples of the preferred sulfonylureas include but are not limited to chlorsulfuron, metsulfuron methyl, ethametsulfuron, methyl tribenuron methyl, thifensulfuron methyl, triflusulfuron methyl, nicosulfuron, rimsulfuron, chlorimuron ethyl, sulfameturon, benzsulfuron methyl, azimsulfuron, and flupyrsulfuron.

Preferred for reasons of higher herbicidal activity, lower cost or ease of synthesis are compounds of Formula I wherein R¹ is unsubstituted benzyl or unsubstituted methylnapthyl.

The most preferred sulfonylureas are:

N-[[(4,6-dimethoxy-2-pyrimidinyl)amino]carbonyl]-3-(ethylsulfonyl)-2-pyridinesulfonamide

methyl 2-[[[[[4-(dimethylamino)-6-(2,2,2-trifluoroethoxy)-1,3,5-triazin-2-yl]amino]carbonyl]amino]sulfonyl]-3-methylbenzoate

ethyl 2-[[[[(4-chloro-6-methoxy-2-pyrimidinyl)amino]carbonyl]amino]-sulfonyl]benzdate

2-[[[[(4,6-dimethoxy-2-pyrimidinyl)amino]carbonyl]amino]sulfonyl]-N,N-dimiethyl-3-pyridinecarboxamide

2-chloro-N-[[(4-methoxy-6-methyl-1,3,5-triazin-2-yl)amino]carbonyl]-benzenesulfonamide

methyl 2-[[[[(4-methoxy-6-methyl)-1,3,5-triazin-2-yl)amino]carbonyl]amino]-sulfonyl]benzoate

methyl 3-[[[[(4-methoxy-6-methyl-1,3,5-triazin-2-yl)amino]carbonyl]amino]-sulfonyl]-2-thiophenecarboxylate

methyl 2-[[[[(4-methoxy-6-methyl-1,3,5-triazin-2-yl)methylamino]carbonyl]-amino]sulfonyl]benzoate

N-[[(4,6-dimethoxypyrimidine-2-yl)amino]carbonyl]-1-methyl-4-(2-methyl-2H-tetrazol-5-yl)-1H-pyrazole-5-sulfonamide

Another embodiment of the invention is a non-aqueous or aqueous formulation comprising as the active ingredient, a compound of Formula I.

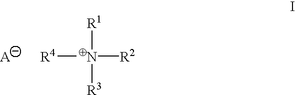

wherein A, R¹, R², R³ are as defined above.

These Formula I compounds are referred to herein as quaternary ammonium salts of Formula I or in the case of the preferred herbicide, sulfonylurea quaternary salts.

The substituted or unsubstituted benzyl or substituted or unsubstituted methylnapthyl group of R¹ is required for chemical stability of the compounds of Formula I and are further defined as the stabilizer group. The substituted or unsubstituted alkyl (C₁-C₂₅) or substituted or unsubstituted alkaryl group of one or more of R², R³ or R⁴ is useful for solubility of the compounds of Formula I in organic solvents or oils. One or more of an alkyl (C₁₀-C₂₅) or alkaryl group of R², R³ or R⁴ is preferred for solubility of compounds of Formula I in organic solvents or oils.

An example of an embodiment of this invention is the stable formulation comprising compounds of Formula I in hydrophobic organic solvents optionally with emulsifiers and other formulation ingredients. Another example of this embodiment is the stable formulation comprising compounds of Formula I in water optionally with surfactants and other formulation ingredients.

Another embodiment of this invention are the stable formulations derived from mixtures of compounds of Formula I with other herbicides like bromoxynil.

In the above recitations, the term “alkyl”, includes straight-chain or branched alkyl, for example, methyl, ethyl, n-propyl, i-propyl, octyl, dodecyl, hexadecyl, octadecyl or, for example, the different butyl, pentyl, or hexyl isomers.

The term “alkoxy” includes, for example, methoxy, ethoxy, n-propyloxy and isopropyloxy.

The term “halogen”, includes fluorine, chlorine, bromine or iodine.

### DETAILED DESCRIPTION OF THE INVENTION

The novel quaternary ammonium salts of Formula I, stabilized aqueous and non-aqueous solution formulations containing these salts and optionally mixtures of said salts of Formula I with other pesticides are prepared in water or organic solvents. Solution formulations are desirable because of the ease with which they can be measured, poured, handled or diluted in preparation for spraying. In addition, the processes and equipment necessary for preparing solution formulations are simpler and less costly than those needed for manufacturing dry formulations or dispersions in a true solution system. Many pesticides including sulfonylurea herbicides are susceptible to the degradative effects of moisture and impurities present even at trace levels in all practical solvent systems. Hence, the storage stability of these formulations may be a limiting factor in their usefulness over any period of time. Due to the high herbicidal activity of the pesticides, e.g. the sulfonylureas, the pesticides may be required in only low concentrations in a formulation which aggravates the problem of stability because of the increased relative concentration of the contaminants which promote decomposition rendering the solutions unstable.

The quaternary ammonium salts of the pesticides of the invention are described herein after with reference to the preferred pesticide which is a sulfonylurea herbicide. However, the invention is directed to such salts of any pesticide with a pKa from about 3 to about 7.

In addition to stability, potential advantages of the sulfonylurea quaternary salt and herbicidal compositions thereof include: 1) better coverage of plant foliage and less wash-off, since the physical form of the sulfonylurea can be altered from a solid, or waxy solid to a liquid form, 2) increased uptake/translocation in the plant, resulting in higher efficacy or broadened spectrum of activity, 3) built-in adjuvancy in a premix formulation, where tank-mixed adjuvants are no longer necessary (e.g., with N-[[(4,6-dimethoxy-2-pyrimdinyl)amino]carbonyl]-3-(ethylsulfonyl)-2-pyridinesulfonamide, 4) allows mixtures with other pesticides in a liquid formulation (e.g., solution), where the efficacy of neither is reduced.

The bioactive sulfonylureas can be prepared by methods known in the art. For instance, U.S. Pat. Nos. 4,127,405 and 4,169,719 disclose herbicidal sulfonylureas, and. Lonza is a commercial source of the quaternary halides which are reacted with the bioactive sulfonylureas as illustrated in the Example to form the compounds of the invention.

Other bioactive pesticides of the invention can be prepared by methods known in the art, e.g. U.S. Pat. Nos. 3,657,443, and 3,799,758, The Hormon Weedkillers, C. Kirby (1980), and U.S. Pat. No. 3,576,834.

Stabilized solution formulations which include the compounds of the invention can be prepared in situ in the desired solvent. First, the inorganic salt of the bioactive sulfonylurea compound is generated by the addition of an inorganic base such as an alkali or alkaline earth hydroxide to a suspension of the bioactive sulfonylurea in the solvent of choice. This is followed by the addition of a quaternary halide. Similarly, one could mix a quaternary hydroxide with a suspension of the bioactive sulfonylurea in the solvent of choice. Compounds of Formula I can also be prepared by exchange of one cation for another, where the side reaction product such as the inorganic halide or water is separated. Cationic exchange can be effected by mixing an aqueous solution of the quaternary halide with an aqueous solution of the inorganic salt of the sulfonylurea. The quaternary sulfonylurea salt is then isolated by filtration (if solid) or extraction with a water immiscible organic solvent of choice. Organic solutions free from inorganic halides are preferably made in situ in the organic solvent of choice followed preferably by filtering off the solid halides. Exchange may also be effected by passing a solution of the inorganic salt of the sulfonylurea through a column packed with a cation exchange resin containing the quaternary cation to be exchanged. In this method, the cation of the resin is exchanged for the inorganic cation of the original sulfonylurea salt, and the quaternary sulfonylurea of Formula I is eluted from the column dissolved in the solvent of choice and free from inorganic halides.

Liquid formulation of sulfonylureas are desirable because of the ease with which they can be measured, poured, handled or diluted in preparing aqueous slurries for spraying. Generally, however, the bioactive sulfonylurea compounds have limited solubility in water and in commercially feasible organic solvents and are chemically unstable upon storage. In addition, the known salts of sulfonylureas have either low solubility in the solvent of choice or are chemical unstable when in solution.

It has been surprisingly found that the sulfonylurea quaternary salts of the invention can be formulated with appreciable solubility in desired solvents along with improved chemical stability. This chemical stabilization is achieved by selecting a benzyl or methylnapthyl group as one of the substituents attached to the quaternary nitrogen. The desired solubility in the solvent of choice is achieved by selecting, in addition to the stabilizing group, short carbon chain groups for water solubility or long carbon chain groups for oil solubility. Judicious choice of the balance of substituent groups on the nitrogen can impart surface activity to the final sulfonylurea quaternary salt.

Preferred nitrogen substituents of R², R³ or R⁴ of Formula I for formulations containing hydrophobic oils are one, two, or preferably three long chain alkyl (C₁₀-C₂₅) or alkaryl groups in addition to the stabilizing group.

Preferred nitrogen substituents of said R², R³ and R⁴ for water soluble formulations are C₁-C₃ alkyl and optionally one alkaryl group in addition to the stabilizing group.

The combination of one or two methyl groups with one or two long chain alkyl or alkaryl groups can impart surface activity to the benzyl or methylnapthyl quaternary salts. These are useful in single premix formulations to impart built in adjuvancy to the sulfonylurea, where separately purchased adjuvants are ordinarily required to be tank mixed to achieve such adjuvancy.

Furthermore, substituent selection can improve herbicidal efficacy or spectrum selectivity by enhancing the solubility of the sulfonylurea quaternary salt in the leaf wax, thereby promoting biotransport into the leaf.

One formulation embodiment of this invention is where the sulfonylurea quaternary compound of the invention is dissolved in a hydrophobic oil, optionally in the presence of a surfactant, and the resulting solution is then applied to the surface of a rice paddy where it spreads to a thin surface layer on the water.

Another formulation embodiment is when an organic solution of the sulfonylurea quaternary compound of the invention in the presence of emulsifiers is formulated as an emulsifiable concentrate (EC). The EC is subsequently mixed in a mix tank with water to form an oil in water emulsion for foliar spray application on crops.

Another formulation embodiment is the sulfonylurea quaternary compound of the invention in an aqueous or organic based gel which can be added to a mix tank of water for foliar spray application.

In some cases, bioactivity is improved by increased solvent solubility (in water or oil) where the sulfonylurea is presented in solution form to the leaf rather than as a particulate e.g. from a dry flowable (DF) or suspension concentrate (SC).

Finally, other bioactivity advantages may be gained by virtue of converting the solid sulfonylurea to a waxy solid, glass or oily liquid even when the solvent is evaporated. The waxy solid, glassy or oily deposit may also improve coverage on plant foliage as well as help prevent wash off.

Another embodiment of the invention includes premix formulations comprising, in addition to the bioactive sulfonylurea quaternary salt, additional herbicides such as bromoxanil and acetochlor. Other herbicides that may be used in the formulations with the bioactive sulfonylurea quaternary salts include a partner herbicide such as a hormonal, anticholine esterase, or glyphosate. Examples of hormonal herbicides include phenoxies, such as (2,4-dichlorophenoxy)acetic acid (2,4D) derivatives and 4-chloro-2-methylphenoxy acetic acid (MCPA). Examples of anticholine esterase herbicides include organophosphorous herbicides such as anilofos. The sulfonylurea quaternary salt and the partner may be dissolved in a common solvent containing emulsifiers and other formulation ingredients to form a premix EC. Preferably, proton scavengers such as epoxides (e.g. epoxidized soybean oil) are added for additional stabilization. Solid, anhydrous calcium acetate can also be added as a suspension to organic liquid formulations to scavenge water and to aid chemical stabilization of the sulfonylurea.

Preferred organic solvents are natural crop oils such as soybean oil, corn oil, cottonseed oil, sunflower oil and epoxidized or methylated versions, propylene carbonate, triethyl phosphate, n-alkyl pyrrolidones, and crop oil esters such as methylsoyate (Henkel) or acetates such as heptyl acetate and Exxates® (Exxon) and mixtures thereof. Hydrophobic oils such as diisodecyl adipate and C₈-C₁₂ alcohols may be used for spreading in rice paddy applications.

The aqueous solution formulations may optionally contain glycols as antifreeze such as propylene glycol.

The organic solution formulations may optionally contain dissolved surfactants at concentrations ranging from 0. 1 to 60%. Among the useful surfactants are common nonionics such as the polyoxyethylene alcohols, tristyrylphenols, nonyl or octyl phenols, esters, diesters, and sorbitol esters, polyoxyethylene/propylene block copolymers, ethoxylated siloxanes, acetylenic diols, and polyglucosides. Anionic surfactants include for example alkylnaphthalene sulfonates, alkylbenzene sulfonates, alpha olefin sulfonates, calcium and ammonium lignosulfonates, dodecylbenzene sulfonates, napthalene/formaldehyde condensates, sulfosuccinates, alkyl and aryl sulfates and phosphates, ethoxylated lignosulfonates, ethoxylated alkyl sulfonates, and ethoxylated di- and tristyrylphenols as the sulfate and phosphate salt.

The following examples are meant to exemplify but not to limit this invention. In all examples, a slight excess (about 5%) of an equivalent amount of reactant can be used. (e.g. NaOH>sulfonylurea>quaternary halide or quaternary hydroxide)

### EXAMPLE 1

Quaternary salt of 2-[[[[(4.6-Dimethoxy-2-Pyrimidinyl)amino]carbonyl]amino]sulfonyl]-N,N-Dimethyl-3-Pyridinecarboxamide

To 21.57 g 2-[[[[(4,6-dimethoxy-2-pyrimidinyl)amino]carbonyl]-amino]sulfonyl]-N,N-dimethyl-3-pyridinecarboxamide and 50 mL 1.0 N NaOH in 1400 mL methylene chloride was incrementally added 22.37 g of diisobutylphenoxy ethoxyethyl dimethylbenzyl ammonium chloride (Hyamine 1622, commercially available from Lonza), with several small methylene chloride washes to facilitate transfer. The reaction medium was filtered through a bed of molecular sieves to remove mineral salts and water. Methylene chloride was stripped to recover the white powder. Upon aging for 1 week at 54 C., 9% relative decomposition of the sulfonylurea resulted as measured by HPLC.

### EXAMPLE 2

In situ Quaternary Salt of Methyl 3-[[[[(4-Methoxy-6-Methyl-1,3,5-Triazin-2-yl)Amino]carbonyl]amino]sulfonyl]-2-Thiophenecarboxylate

Into 7.8 g of epoxidized soybean oil (ESBO) was added with stirring: 1.18 g methyl 3-[[[[(4-methoxy-6-methyl-1,3,5-triazin-2-yl)amino]carbonyl]amino]sulfonyl]-2-thiophenecarboxylate tech (98.1%), 1.31 g of anhydrous diisobutylphenoxy ethoxyethyl dimethylbenzyl ammonium chloride in H₂O (Hyamine 1622, available from Lonza), 0.11 g Ca(OH)₂, and 0.03 g calcium acetate. The mixture was aged 3 weeks at 45° C. giving 0% relative decomposition of the sulfonylurea as measured by HPLC.

### EXAMPLE 3

Quaternary Salt of Methyl 2-[[[[(4-Methoxy-6-Methyl-1,3,5-Triazin-0.2-yl)Methylamino]carbonyl]amino]sulfonyl]benzoate

A solution on 15 g of methyl 2-[[[[(4-methoxy-6-methyl-1,3,5-triazin-2-yl)methylamino]carbonyl]amino]sulfonyl]benzoate tech (95.7%) and 18 g of Na₂CO₃ in 300 g of CH₂Cl₂ was treated with 1.5 g of NaOH and stirred 2 h. 8.65 g of benzyl triethyl ammonium chloride was added to the suspension and the mixture stirred overnight. The suspension was filtered and the filtrate stripped of solvent on a rotary evaporator yielding the corresponding sulfonylurea quaternary salt as a viscous yellow oil (15.7 g) using 1 g of the oil, a 20% solution of the quaternary salt was made in triethyl phosphate. Upon aging for 1 week at 54° C., 1% relative decomposition of the sulfonylurea resulted as indicated by HPLC.

### EXAMPLE 4

Quaternary Salt of Methyl 2-[[[[(4-Methoxy-6-Methyl-1,3,5-Triazin-2-yl)Methylamino]carbonyl]amino]sulfonyl]benzoate

A quaternary salt sulfonylurea was prepared using the procedure of Example 3 with 20 g methyl 2-[[[[(4-methoxy-6-methyl-1,3,5-triazin-2-yl)methylamino]carbonyl]-amino]sulfonyl]benzoate, 30 g Na₂CO₃, 400 g CH₂Cl₂, 2 g NaOH and 11.5 g benzyl triethyl ammonium chloride. One gram of the resulting orange viscous oil was made up as a 20% solution in triethyl phosphate. Upon aging for 1 week at 54° C., 2% relative decomposition of the sulfonylurea resulted as indicated by HPLC.

### COMPARATIVE EXAMPLE A

### Control for Oil Soluble Sulfonylurea Quaternary Salt

Tetradodecyl ammonium bromide was dissolved in 5 mL of CH₂Cl₂ and stirred with 3.3 mL of 1 N NaOH. To this was added 1.27 g of N-[[(4,6-dimethoxypyrimidine-2-yl)amino]carbonyl]-1-methyl-4-(2-methyl-2H-tetrazol-5-yl)-H-pyrazole-5-sulfonamide (97.8%) dissolved in 5 mL of CH₂Cl₂ and stirring continued for 5 min at 25° C. The organic phase was separated, washed with water, dried and the solvent removed tinder vacuum in a rotary evaporator at a maximum of 50° C. The resulting viscous, colorless oil gave an assay of 35% of the corresponding sulfonylurea quaternary salt (vs. 38% theory) using HPLC. This oil was more than-50% soluble in epoxidized soybean oil, methyl caprylate/caprate, and cottonseed oil. 50% solutions of the resultant sulfonylurea quaternary salt in these three solvents were aged 1 week at 54° C., giving 20-45% relative degradation by HPLC analysis. The N-[[(4,6-dimethoxypyrimidine-2-yl)amino]carbonyl]-1-methyl-4-(2-methyl-2H-tetrazol-5-yl )-1H-pyrazole-5-sulfonamide was practically insoluble in these three solvents.

### COMPARATIVE EXAMPLE B

### Control for Water Soluble Sulfonylurea Quaternary Salt

In a vial was vortexed the following: 0.214 g methyl 2-[[[[[(4,6-dimethoxy-2-pyrimidinyl)amino]carbonyl]amino]sulfonyl]methyl]benzoate (98.3%), 0.192 g of a 25% aqueous solution of tetramethyl ammonium hydroxide, and 4.32 g of water to produce the sulfonylurea quaternary salt. Upon aging the solution at 54° C. for one week, 52% degradation of the sulfonylurea resulted as measured by HPLC.

### COMPARATIVE EXAMPLE C

### Control Using Non-Salt of a Sulfonylurea in Water

Deionized water (5 g) was saturated with 2-[[[[(4,6-dimethoxy-2-pyrimidinyl)amino]carbonyl]amino]sulfonyl]-N,N-dimethyl-3-pyridinencarboxamide tech (500 ppm) and aged at 25° C. for 30 days. HPLC analysis indicated that 90% relative degradation of the sulfonylurea resulted.

What is claimed is:

1. Compounds of the Formula I 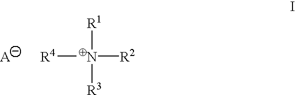
where A is any pesticidally active compound with oKa from about 3to about 7; R¹ is substituted or unsubstituted benzyl or substituted or unsubstituted methylnapthyl group; R², R³ and R⁴ are each a substituted or unsubstituted alkyl or substituted or unsubstituted alkaryl group, provided that when the pesticide A is a sulfonylurea herbicide of the formula JSO₂NHCONR⁶Q and R¹ is unsubstituted benzyl, then one of R², R³ or R⁴ is other than methyl or ethyl;

2. The compounds of claim 1 wherein the pesticidally active compound A is a herbicidal sulfonylurea compound generally represented by the formula J SO₂NH CONR⁶ Q where J is a substituted or unsubstituted phenyl or heterocyclic group, Q is a substituted or unsubstituted pyrimidine or triazine group and R⁶ is H or methyl.

3. The compounds of claim 2 wherein R¹ is an unsubstituted benzyl group

4. The compounds of claim 3 wherein R², R³, R⁴ are C₁₀-C₂₅ alkyl or an alkaryl group.

5. The compounds of claim 2 wherein R¹ is a substituted benzyl group

6. The compounds of claim 5 wherein R², R³, R⁴ are C₁₀-C₂₅ alkyl or an alkaryl group.

7. A herbicidal composition for controlling the growth of undesired vegetation which comprises an effective amount of a compound of claim 1 and at least one of the following: surfactant or liquid inert diluent.

8. A herbicidal composition for controlling the growth of undesired vegetation which comprises an effective amount of a compound of claim 2 and at least one of the following: surfactant or liquid inert diluent.

9. A herbicidal composition for controlling the growth of undesired vegetation which comprises an effective amount of a compound of claim 3 and at least one of the following: surfactant or liquid inert diluent.

10. A herbicidal composition for controlling the growth of undesired vegetation which comprises an effective amount of a compound of claim 3 and at least one of the following: surfactant or liquid inert diluent, wherein the surfactant is a tristyrylphenol or octylphenol and the liquid inert diluent is water, epoxidized soybean oil or triethylphosphate.

11. A method for controlling the growth of undesired vegetation which comprises applying to the locus to be protected an effective amount of a compound of claim 1.
